(19) United States
(12) Patent Application Publication
(10) Pub. No.: US 2005/0256061 A1
(43) Pub. Date: Nov. 17, 2005

(54) Use of isoflavones for preparing topical compositions for promoting slimming, and related cosmetic treatment method

(75) Inventors: Philippe Msika, Versailles (FR); Nathalie Piccardi, Saint Egreve (FR)

Correspondence Address: FOLEY AND LARDNER;SUITE 500, 3000 K STREET NW, WASHINGTON, DC, 20007, US

(73) Assignee: Laboratoires Expanscience

(21) Appl. No.: 10/519,096

(22) PCT Filed: Jun. 25, 2003
(86) PCT No.: PCT/FR03/01956

§ 371 Date: Dec. 27, 2004

(30) Foreign Application Priority Data
Jun. 27, 2002 (FR) 0207995

(51) Int. Cl. A61K 31/7048; A61K 31/353

(52) U.S. Cl. 514/27; 514/456

(57) ABSTRACT

The invention concerns the use of isoflavones for ping topical compositions for promoting slimming, and in particular for fighting against localized excess weight; the related cosmetic treatment method and a composition comprising additionally to isoflavones an extract of Sophora Japonica flowers.

Use of isoflavones for preparing topical compositions for promoting slimming, and related cosmetic treatment method.

The present invention relates to the use of isoflavones for the preparation of topical compositions that are useful for promoting slimming, and to the related cosmetic treatment method.

In the context of the present invention, slimming is preferentially a case of fighting localized excess weight.

This localized excess weight appears in the form of fat, the amount and distribution of which differ according to the sex. Thus, adipose tissue represents 20 to 30% of the body weight in women and 10 to 15% in men. Subcutaneous fat is twice as thick in women as in men. In men, fat accumulates around and above the waistline (android distribution, metabolic risk factor) and below the waistline, in the gluteo-femoral region in women (gynoid distribution, not correlated with a vascular risk). One of the characteristics of this accumulated lower body fat is that it is difficult to mobilize. It is intended to ensure the energetic needs of reproduction (pregnancy and, above all, breastfeeding) and thus constitutes the largest energy reservoir in the body.

At the cellular level, adipocytes are spherical cells whose intracellular space is occupied by a large vacuole filled with triglycerides. The adipocytes can change rapidly in volume. Specifically, depending on the circumstances, these cells may reach 40 μm to 120 μm in diameter, which corresponds to a 27-fold increase in volume. In certain extreme cases, this increase may be up to 40-fold. Thus, the adipocyte is the main energy factor in the body since it is capable of rapidly storing (uptake or lipogenesis) or, conversely, mobilizing (lipolysis) the triglycerides, which are the main energy sources of the body.

Lipogenesis proceeds via the synthesis of triacylglycerols, which result from the esterification of glycerol 3-phosphate with activated fatty acids; conversely lipolysis corresponds to the hydrolysis of the stored triacylglycerols, to glycerol and fatty acids. Various mechanisms have been brought to light, which control lipolysis and lipogenesis and involve, for example, receptors such as the alpha-2 and/or beta-1 and -2 receptors, the type A1 adenosine receptors, the prostaglandin E2, Y2 of YY type and the neuropeptide NPY receptors, but also sexual hormones.

Thus, the knowledge of the mechanisms controlling adipocyte lipolysis and lipogenesis has been very markedly improved. However, slimming active agents are still sought, since the known slimming active agents are not entirely satisfactory. There is thus at the present time a genuine demand to develop topical compositions for efficiently promoting slimming.

Two major types of slimming active agents are known: lipolytic agents (acting on the removal of the excess lipids) and liporeducing agents (combating the formation of fat).

a) Lipolytic Agents

Caffeine (which is found in many plants: green tea, guarana seeds): inhibits phosphodiesterase, thus ensuring an optimum intracellular level of cAMP, stimulates the β receptors and inhibits the lipoprotein lipase;
rhodysterol (extract of a red alga): activates the α receptors and promotes the penetration of caffeine;
palmitoyl-carnitine: accelerates the combustion of fatty acids, by improving their uptake by the mitochondria;
alpha and gamma bioactive agents (derived, respectively, from a marine bacterium and a fungus): block the α2 and NPY receptors;
escin and ginkgo biloba: α2 blockers;
sphingosine: limits the penetration of glucose;
   b) Liporeducing Agents
Andiroba (triterpenes) and Carcina cambogia: block the conversion of the preadipocytes into adipocytes;
rutin: (extracted from Ruta graveolens): isolates glucose and prevents it from combining with free fatty acids.

To these specific active agents may be added anti-infiltration active agents and venotonic agents, which are often combined with slimming active agents.

Anti-Infiltration Active Agents:

Viburnum (draining, decongesting, active caffeine effect, free-radical scavenging, firming)
Anti-inflammatory, anti-edematous, analgesic
Arnica (anti-edematous, calmative)
Mouseear hawkweed (free-radical scavenging, accelerates draining)
Wild pansy (important role in water balance)
Fucus vesiculosus (anti-edematous, calmative)
   Venotonic Agents:
Ruscus (vitamin P action, vascular tonic)+
Ginkgo biloba (fights vascular and capillary stasis)
Escin (improvement in venous tonus, modifies the capillary permeability)

Finally, formulations comprising these known slimming active agents can be complemented with restructuring and smoothing active agents that fight sagging of the skin.

It has now been found that the application of a topical composition comprising one or more isoflavones has a slimming action and makes it possible especially to fight localized excess weight.

Patent application WO 01/64177 (Henkel KGAA) discloses the use of a composition comprising a flavone, an isoflavone, or glycosyl and optionally a substance for stimulating and/or depolarizing the nerve fibers, for the cosmetic treatment of cellulite or firming of the skin.

In the article Cosmetics, D. Schmid et al., SÖFW-Journal, 127. Jahrgang 10-200, “Genisteine, a new cosmetic ingredient derived from soy”, the anti-cellulite action of certain isoflavones is described. However, cellulite is a condition that is quite different from localized excess weight.

Thus, cellulite is defined as a localized metabolic disorder of the subcutaneous tissues that causes an impairment in the shape of the body. Conversely, excess weight is caused by hypertrophy or hyperplasia of the adipocytes (increase in lipogenesis), or a reduction in lipolysis. The cellulite-slimming differentiation also appears in view of the fact that cellulite affects the dermis (subcutaneous tissue) of women only, whereas excess weight (the adipocytes) affects both sexes. Furthermore, cellulite appears in women even without excess weight; it is a problem of dermal/hypodermal architecture.

Patent application EP 829 261 relates to compositions comprising isoflavones for their weight-reducing effect via the degradation of fat accumulated in the fat cells. However, the topical mode of administration is not mentioned in said patent application.

One subject of the present invention is thus the use of isoflavones for the preparation of topical compositions that are useful for promoting slimming, and the related cosmetic treatment method.

The “isoflavones” that may be used according to the present invention may be natural substances extracted from natural products, especially from plants such as soybean, clover, lupin, apple seeds, etc. The topical compositions according to the present invention quite often contain, as isoflavones, a mixture of different isoflavones, but they may also be present in pure form in the context of the present invention. Moreover, the aglycone forms of the isoflavones and the glycosylated forms thereof are distinguished. These various forms are usually found as a mixture. They are illustrated by the following formulae.

Aglycone forms, of formula: 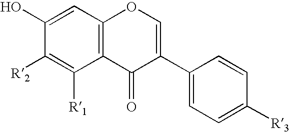
in which R′₁ represents a hydrogen atom or a hydroxyl group, R′₂ represents a hydrogen atom or a methoxy group and R′₃ represents a hydroxyl group.

Advantageously, according to the present invention, R′₁, R′₂ and R′₃ represent:

| R′₁ | R′₂ | R′₃ | Compound name |
|---|---|---|---|
| H | H | OH | daidzein |
| OH | H | OH | genistein |
| H | OCH₃ | OH | glycitein |

Glycosylated forms, of formula: 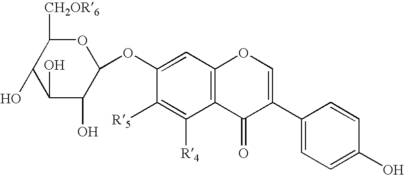
in which R′₄ represents a hydrogen atom or a hydroxyl group, R′₅ represents a hydrogen atom or a methoxy group and R′₆ represents a hydrogen atom.

Advantageously, according to the present invention, R′₄, R′₅ and R′₆ represent:

| R′₄ | R′₅ | R′₆ | Compound name |
|---|---|---|---|
| H | H | H | daidzine |
| OH | H | H | genistine |
| H | OCH₃ | H | glycitine |

The glycosylated forms of the isoflavones are the ones that are the most abundant in nature.

The isoflavones that are preferred are the natural isoflavones such as genistein (1), daidzein or glycitein.

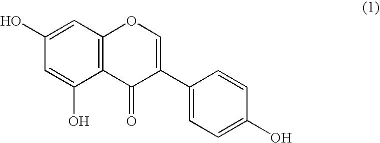

In particular, genistein, or 4,5,7-trihydroxy-isoflavone, which may be used according to the present invention may be a product of plant origin and especially from soybean, with a titer of 85% to 90% by weight of genistein, especially the product sold by the company Buckton Scott under the name “85%-titer genistein”.

The isoflavones may be used alone or as a mixture in the context of the present invention.

The topical application of a composition of one or more isoflavones may prove to be particularly advantageous in the case of pregnant women or women who have given birth within 6 months. A subject of the present invention is thus also a cosmetic treatment method for promoting slimming and especially for fighting localized excess weight in pregnant women or women who have given birth within 6 months. Specifically, one of the advantages of the compositions that may be used in the context of the present invention is that the presence of alcohol, which is contraindicated for pregnant and breastfeeding women on account of its toxicity, is not necessary. Specifically, caffeine, a very commonly used slimming agent, requires dissolution in alcohol, which is thus avoided in the context of the present invention.

According to the present invention, the terms “slimming” and “fighting localized excess weight” mean an action making it possible to avoid or at the very least reduce the formation of subcutaneous fat as described previously. This action is especially reflected by a reduction in unsightly excesses or reserves, by refining the silhouette, by accelerating the removal of excesses, and by a better definition of the contour of the body or alternatively a resculptured silhouette.

According to the present invention, the expression “cosmetic treatment method for fighting localized excess weight” means the use of a cosmetic treatment that makes it possible to visibly measure the action described above.

Thus, a topical composition comprising one or more isoflavones used according to the invention may be applied to the areas of skin liable to form this localized excess weight, namely areas where these excesses have already formed or are in the process of being formed.

By way of example, a composition containing genistein that may be used in the context of the present invention may contain between 0.0085% and 8.5% of genistein by weight relative to the total weight of the composition, i.e. for a solution with an 85% to 90% by weight titer of genistein, between 0.01% and 10% by weight of this solution relative to the total weight of the composition.

More generally, a composition containing one or more isoflavones that may be used in the context of the present invention may contain between 0.01% and 10%, and preferably from 0.1% to 3%, by weight of isoflavone(s) relative to the total weight of the composition.

The composition that allows the invention to be implemented comprises a cosmetically acceptable support, i.e. a support that is compatible with the skin, and may be in any galenical form normally used for topical application, especially in the form of an aqueous, aqueous-alcoholic or oily solution, an oil-in-water or water-in-oil or multiple emulsion, an aqueous or oily gel, a liquid, pasty or solid anhydrous product, a dispersion of oil in an aqueous phase with the aid of spherules, these spherules possibly being polymer nanoparticles such as nanospheres and nanocapsules or better still lipid vesicles of ionic and nonionic type.

This composition may be more or less fluid and may have the appearance of a white or colored cream, an ointment, a milk, a lotion, a serum, a paste, a mousse or a gel.

It may optionally be applied to the skin in the form of an aerosol. It may also be in solid form, for example in the form of a stick.

The composition of the invention may also contain adjuvants that are common in cosmetics, such as hydrophilic or lipophilic gelling agents, hydrophilic or lipophilic active agents, preserving agents, antioxidants, solvents, fragrances, chelating agents, odor absorbers and dyestuffs. The amounts of these various adjuvants are those conventionally used in the fields under consideration, for example from 0.01% to 20% of the total weight of the composition. Depending on their nature, these adjuvants may be introduced into the fatty phase, into the aqueous phase, into lipid vesicles or into nanoparticles.

When the composition of the invention is an emulsion, the proportion of the fatty phase may range from 5% to 80% and preferably from 5% to 50% by weight relative to the total weight of the composition. The oils, emulsifiers and coemulsifiers used in the composition in emulsion form are chosen from those conventionally used in the field under consideration. The emulsifier and coemulsifier are present in the composition in a proportion ranging from 0.3% to 30% and preferably from 0.5% to 20% by weight relative to the total weight of the composition.

As oils that may be used in the compositions for implementing the invention, mention may be made of mineral oils, oils of plant origin (apricot oil, sunflower oil or plum oil), oils of animal origin, synthetic oils, silicone oils and fluoro oils (perfluoropolyethers). Fatty alcohols (cetyl alcohol), fatty acids and waxes (beeswax) may also be used as fatty substances.

As emulsifiers and coemulsifiers that may be used in the invention, examples that may be mentioned include fatty acid esters of polyethylene glycol, such as PEG-40 stearate and PEG-100 stearate, and fatty acid esters of polyols, such as glyceryl stearate and sorbitan tristearate.

Hydrophilic gelling agents that may be mentioned in particular include carboxyvinyl polymers (carbomer), acrylic copolymers such as acrylate/alkylacrylate copolymers, polyacrylamides, polysaccharides, natural gums and clays, and lipophilic gelling agents that may be mentioned include modified clays, for instance bentones, metal salts of fatty acids, hydrophobic silica and polyethylenes.

The composition used according to the invention may contain other active agents with a slimming action, for instance the lipolytic agents and liporeducing agents as described in the introduction.

The invention thus relates to the use of isoflavones for the preparation of topical compositions that are useful for promoting slimming and especially for fighting localized excess weight, characterized in that one or more isoflavones and also one or two slimming active agents of lipolytic type and/or one or two slimming active agents of liporeducing type are applied, simultaneously, separately or sequentially over time.

The slimming active agent of lipolytic type may be chosen from: caffeine, rhodysterol, palmitoylcarnitine, alpha and gamma bioactive agents, escin, ginkgo biloba and sphingosine. The slimming active agent of liporeducing type may be chosen from: andiroba, Garcinia cambogia and rutin.

One or two anti-infiltration or venotonic active agents may also be applied simultaneously, separately or sequentially over time in addition to the application of the composition used according to the invention. The anti-infiltration or venotonic active agents may be chosen from: viburnum, ivy, arnica, mouseear hawkweed, wild pansy, Fucus vesiculosus, Ruscus, ginkgo biloba and escin.

The composition used according to the invention may also comprise other active agents, such as:
an extract of Sophora japonica flowers: this extract is rich in flavanoids (free-radical-scavenging) and in rutin. This active agent promotes the capillary circulation, facilitating and activating the draining and anti-infiltrations of tissues;
extract of Centella asiatica: extract of Centella, a plant originating from east Africa and from Madagascar. This active agent contains terpenes (asiaticosides, asiatic acid and madecassic acid) with draining, anti-infiltration and firming properties on tissues. It is especially used in slimming products, but also in anti-stretch mark, anti-wrinkle and siccatrizing products; 0 to 5% extract of Centella may thus be present in a slimming composition;
“Hydrolyzed Soy Protein”: soy protein, which is an elastoregulator. These soy peptides may be any peptide obtained by hydrolysis of proteins extracted from soy, according to operating conditions known to those skilled in the art, in other words any soy protein hydrolysate. Soy peptides, which are described in patent application WO 00/19974, are particularly suitable for introduction into the compositions used in the context of the present invention. This active agent allows restoration of the mechanisms of cell renewal, activates the synthesis of the structural components of the extracellular matrix and has a restructuring, regenerating and firming action; 0 to 5% soy protein may thus be present in a slimming composition;
anti-wrinkle active agents, among which mention may be made of avocado furans, retinol and its derivatives, vitamin C or unsaponifiable matter from soy, etc.

The combination of the two active agents: isoflavone taken alone or as a mixture, and extract of Sophora japonica flowers, is particularly preferred and a composition containing this combination forms part of the invention. Such a composition may contain from 0.01% to 20% of extract of Sophora japonica flowers.

The examples that follow illustrate the present invention.

### EXAMPLE 1

### Restructuring Gel

| Ingredients (INCI - EU) | % w/w |
|---|---|
| Aqua | qsp |
| PEG-6 | 3.6000 |
| Butylene glycol | 2.7000 |
| Dextrin | 1.8600 |
| Phenyl trimethicone | 1.2000 |
| Acrylates/C10-30 alkyl acrylate | 0.6000 |
| crosspolymer | |
| Preservative system | qsp |
| Dimethicone/phenyl vinyl | 0.3000 |
| dimethicone crosspolymer | |
| Fragrance | qs |
| Sophora japonica flower extract | 0.01 to 20 |
| Xanthan gum | 0.1500 |
| PPG 26-Buteth-26 | 0.1100 |
| Genosten ® 4000⁽¹⁾ | 1 to 10 |
| Hydrolyzed soy protein | 0.1000 |
| Glucose | 0.0800 |
| PEG 40 hydrogenated castor oil | 0.0700 |
| Sorbitol | 0.0400 |
| Centella asiatica extract | 0.01 to 5 |
| Citric acid | 0.0200 |
| Enteromorpha compressa extract | 0.01 to 5 |
| Colorant | qs |
| Sodium hydroxide | qsp pH = 4.5 to 6.5 |

qs = quantity sufficient
qsp = quantity sufficient for
⁽¹⁾sold by the company Nutrinov

Isoflavone-rich soy extract obtained by physical extraction:

4% isoflavones including:

| | | |
|---|---|---|
| Daidzine: | 0.28 | g |
| O. Malonyl daidzine: | 0.93 | g |
| Genistine: | 0.28 | g |
| O. Malonyl genistine: | 2.50 | g |
| Genistein: | 0.02 | g |
| Daidzein: | 0 | g |

### EXAMPLE 2

### Restucturing Gel

| Ingredients (INCI - EU) | %w/w |
|---|---|
| Aqua | qsp |
| PEG-6 | 3.6000 |
| Butylene glycol | 2.7000 |
| Dextrin | 1.8600 |
| Phenyl trimethicone | 1.2000 |
| Acrylates/C10-30 alkyl acrylate | 0.6000 |
| crosspolymer | |
| Preservative system | qsp |
| Dimethicone/phenyl vinyl | 0.3000 |
| dimethicone crosspolymer | |
| Fragrance | qs |
| Sophora japonica flower extract | 0.01 to 20 |
| Xanthan gum | 0.1500 |
| PPG 26-Buteth-26 | 0.1100 |
| 4,5,7-trihydroxyisoflavone | 0.01 to 10 |
| Hydrolyzed soy protein | 0.1000 |
| Glucose | 0.0800 |
| PEG 40 hydrogenated castor oil | 0.0700 |
| Sorbitol | 0.0400 |
| Centella asiatica extract | 0.01 to 5 |
| Citric acid | 0.0200 |
| Enteromorpha compressa extract | 0.01 to 5 |
| Colorant | qs |
| Sodium hydroxide | qsp pH = 4.5 to 6.5 |

qs = quantity sufficient
qsp = quantity sufficient for

### EXAMPLE 3

### Oil-in-Water Cream

| Ingredients (INCI - EU) | % w/w |
|---|---|
| Aqua | qsp |
| Squalane | 5.00 |
| Petrolatum | 5.00 |
| Glycerin | 5.00 |
| Isodecyl neopentanoate | 5.00 |
| Pentaerythrityl | 5.00 |
| tetraethylhexanoate | |
| Cyclomethicone | 4.00 |
| Cetearyl alcohol | 3.00 |
| Myristyl myristate | 2.00 |
| Laureth-23 | 2.00 |
| Silica | 2.00 |
| Heptadecadienyl furan | 0.1 to 10 |
| Beeswax | 1.00 |
| Sclerotium gum | 1.00 |
| PEG-6 | 1.00 |
| Polyacrylamide | 0.80 |
| Glyceryl stearate | 0.70 |
| Dimethiconol | 0.70 |
| Cetearyl glucoside | 0.60 |
| C13-14 isoparaffin | 0.40 |
| Citric acid | 0.14 |
| Laureth-7 | 0.10 |
| Quimdis mixture⁽²⁾ | 0.01 to 10 |
| Caffeine | 0.1 to 10 |
| Enteromorpha compressa extract | 0.01 to 5 |
| Garcinia cambogia extract | 0.01 to 10 |
| Ginkgo biloba extract | 0.01 to 10 |
| Sophora japonica flower extract | 0.01 to 20 |
| Hydrolyzed soy protein | 0.01 to 10 |
| Preservative system | qs |
| Fragrance | qs |

qs = quantity sufficient
qsp = quantity sufficient for
⁽²⁾sold by the company Quimdis

Mixture of isoflavones between 7 and 17%
4 to 8% daidzein glycosides
2 to 5% glycitine glycosides
1 to 4% genistein glycosides

### EXAMPLE 4

### Oil-in-Water Cream

| Ingredients (INCI - EU) | % w/w |
|---|---|
| Aqua | qsp |
| Squalane | 5.00 |
| Petrolatum | 5.00 |
| Glycerin | 5.00 |
| Isodecyl neopentanoate | 5.00 |
| Pentaerythrityl | 5.00 |
| tetraethylhexanoate | |
| Cyclomethicone | 4.00 |
| Cetearyl alcohol | 3.00 |
| Myristyl myristate | 2.00 |
| Laureth-23 | 2.00 |
| Silica | 2.00 |
| Heptadecadienyl furan | 0.1 to 10 |
| Beeswax | 1.00 |
| Sclerotium gum | 1.00 |
| PEG-6 | 1.00 |
| Polyacrylamide | 0.80 |
| Glyceryl stearate | 0.70 |
| Dimethiconol | 0.70 |
| Cetearyl glucoside | 0.60 |
| C13-14 isoparaffin | 0.40 |
| Citric acid | 0.14 |
| Laureth-7 | 0.10 |
| 4,5,7-Trihydroxyisoflavone | 0.01 to 10 |
| Caffeine | 0.1 to 10 |
| Enteromorpha compressa extract | 0.01 to 5 |
| Garcinia cambogia extract | 0.01 to 10 |
| Ginkgo biloba extract | 0.01 to 10 |
| Sophora japonica flower extract | 0.01 to 20 |
| Hydrolyzed soy protein | 0.01 to 10 |
| Preservative system | qs |
| Fragrance | qs |

qs = quantity sufficient
qsp = quantity sufficient for

### EXAMPLE 5

### Water-in-Oil Cream

| Ingredients (INCI - EU) | % w/w |
|---|---|
| Aqua | qsp |
| Hydrogenated polyisobutene | 7.00 |
| Isocetyl stearate | 7.00 |
| Cyclomethicone | 4.80 |
| Glycerin | 4.00 |
| Mineral oil | 3.00 |
| Zinc oxide | 3.00 |
| Butylene glycol | 2.00 |
| Isononyl isononanoate | 2.00 |
| Beeswax | 2.00 |
| Cetyl dimethicone copolyol | 1.70 |
| Polyglyceryl-4 isostearate | 1.65 |
| Hexyl laurate | 1.65 |
| Disodium tartrate | 1.60 |
| Sodium chloride | 1.00 |
| PEG-6 | 1.00 |
| 4,5,7-Trihydroxyisoflavone | 0.01 to 10 |
| Retinyl palmitate | 0.01 to 10 |
| Enteromorpha compressa extract | 0.01 to 5 |
| Sophora japonica flower extract | 0.01 to 20 |
| Centella asiatica extract | 0.01 to 5 |
| Hydrolyzed soy protein | 0.01 to 10 |
| Preservative system | qs |
| Fragrance | qs |

qs = quantity sufficient
qsp = quantity sufficient for

### EXAMPLE 6

### Lipstick

| Ingredients (INCI - EU) | % w/w |
|---|---|
| Castor oil | qsp |
| Oleyl alcohol | 20.00 |
| Hydrogenated palm kernel oil | 17.00 |
| Candelilla wax | 11.00 |
| Polyglyceryl-3 beeswax | 10.00 |
| Mineral oil | 9.57 |
| Heptadecadienyl furan | 0.1 to 1 |
| 4,5,7-Trihydroxyisoflavone | 0.01 to 1.10 |
| quaternium-18 hectorite | |
| Titanium dioxide | 1.00 |
| Tocopheryl acetate | 0.50 |
| Propylene carbonate | 0.33 |
| Fragrance | qs |
| Retinol | 0.01 |
| Enteromorpha compressa extract | 0.01 to 5 |
| Sophora japonica flower extract | 0.01 to 20 |
| Centella asiatica extract | 0.01 to 5 |
| Hydrolyzed soy protein | 0.01 to 10 |

qs = quantity sufficient
qsp = quantity sufficient for

### EXAMPLE 7

### Cream Gel

| Ingredients (INCI - EU) | % w/w |
|---|---|
| Aqua | qsp |
| Cyclomethicone | 5.40 |
| Octyl palmitate | 5.00 |
| Hydrogenated cocoglycerides | 3.00 |
| Arachidyl behenyl alcohol | 2.55 |
| Propylene glycol | 2.50 |
| Isodecyl neopentanoate | 2.00 |
| Glyceryl stearate | 1.70 |
| Cetyl alcohol | 1.30 |
| Stearic acid | 1.00 |
| PEG-6 | 1.00 |
| Beeswax | 0.40 |
| C13-14 isoparaffin | 0.40 |
| Butylene glycol | 0.16 |
| Glycerin | 0.16 |
| Cetearyl alcohol | 0.10 |
| Cetyl palmitate | 0.10 |
| Cocoglycerides | 0.10 |
| Laureth-7 | 0.10 |
| Novasoy mixture⁽³⁾ | 0.01 to 10 |
| Enteromorpha compressa extract | 0.01 to 5 |
| Sophora japonica flower extract | 0.01 to 20 |
| Centella asiatica extract | 0.01 to 5 |
| Hydrolyzed soy protein | 0.01 to 10 |
| Preservative system | qs |
| Fragrance | qs |

qs = quantity sufficient
qsp = quantity sufficient for
⁽³⁾sold by the company ADM

Mixture of isoflavones at 30% minimum
Genistein/diadzine/glycitine ratio: 1.3/1.0/0.3

Dosage example:

| | |
|---|---|
| genistein: | 20.80% |
| glycitine: | 3.80% |
| daidzein: | 12.00% |

### EXAMPLE 8

### Cream Gel

| Ingredients (INCI - EU) | % w/w |
|---|---|
| Aqua | qsp |
| Cyclomethicone | 5.40 |
| Octyl palmitate | 5.00 |
| Hydrogenated cocoglycerides | 3.00 |
| Arachidyl behenyl alcohol | 2.55 |
| Propylene glycol | 2.50 |
| Isodecyl neopentanoate | 2.00 |
| Glyceryl stearate | 1.70 |
| Cetyl alcohol | 1.30 |
| Stearic acid | 1.00 |
| PEG-6 | 1.00 |
| Beeswax | 0.40 |
| C13-14 isoparaffin | 0.40 |
| Butylene glycol | 0.16 |
| Glycerin | 0.16 |
| Cetearyl alcohol | 0.10 |
| Cetyl palmitate | 0.10 |
| Cocoglycerides | 0.10 |
| Laureth-7 | 0.10 |
| 4,5,7-Trihydroxyisoflavone | 0.01 to 10 |
| Enteromorpha compressa extract | 0.01 to 5 |
| Sophora japonica flower extract | 0.01 to 20 |
| Centella asiatica extract | 0.01 to 5 |
| Hydrolyzed soy protein | 0.01 to 10 |
| Preservative system | qs |
| Fragrance | qs |

qs = quantity sufficient
qsp = quantity sufficient for

### EXAMPLE 9

### Spray

| Ingredients (INCI - EU) | % w/w |
|---|---|
| Aqua | qsp |
| Glycerin | 4.00 |
| Montmorillonite | 3.00 |
| PEG | 3.00 |
| Glycine | 0.30 |
| Citric acid | 0.09 |
| 4,5,7-Trihydroxyisoflavone | 0.01 to 10 |
| Enteromorpha compressa extract | 0.01 to 5 |
| Sophora japonica flower extract | 0.01 to 20 |
| Centella asiatica extract | 0.01 to 5 |
| Hydrolyzed soy protein | 0.01 to 10 |
| Preservative system | qs |
| Fragrance | qs |

qs = quantity sufficient
qsp = quantity sufficient for

### EXAMPLE 10

### Summary of the Test of Use of the Restructuring Gel According to Example 1

It was sought in this test to assess the efficacy and acceptability of the restructuring gel according to example 1, after repeated applications, twice a day over a period of 8 weeks, under the normal conditions of use, by a group of 92 to 100 consumers who had given birth within 3 months and/or whose body skin (midriff and thighs) had excess fat.

Panelists

The analysis of the results related to a panel of 92 (T 2 months) to 100 (T 1 month) consumers, the physical characteristics of which are given below.

Panel at T 1 Month (100 Consumers)

Panel at T 1 month (100 consumers)

| | | “Sensitive” |
|---|---|---|
| Age | Nature of the body skin | skin |
| 19 to 49 years old | dry: 44% | 35% |
| (average: 30 years | normal: 36% | |
| old) | dry-normal combination: 20% | |

It should be noted that 48 panelists had just been through their first pregnancy, 44 had had previous pregnancies numbering 2 (23), 3 (20) and 4 (1).

Panel at T 2 Months (92 Consumers)

| | | “Sensitive” |
|---|---|---|
| Age | Nature of the body skin | skin |
| 19 to 49 years old | dry: 45% | 37% |
| (average: 30 years old) | normal: 33.7% | |
| | dry-normal combination: 20% | |

It should be noted that 48 panelists had just been through their first pregnancy, and that 40 had had previous pregnancies numbering 2 (21) or 3 (19).

Protocol

The restructuring gel studied was applied twice a day on average (morning and evening), for 1 to 2 months, to the body (thighs, hips, midriff and buttocks in particular), by the consumers, at home, under the normal conditions of use, instead of the product they generally used.

The questionnaires, adapted to the nature of the product, were filled in at the end of the test (T1 and T2 months) and then mailed in, and especially included the questions listed below relating to the action of fighting excess weight.

The restructuring gel according to example 1, applied for 2 months by the 92 to 100 women described above was found particularly favorable, since 87% of the panelists judged it as overall “pleasant” to “very pleasant” and 67% judged it as overall “good” to “very good”. The performance characteristics below were demonstrated.

| | Effects at T 1 month | Effects at T 2 months |
|---|---|---|
| | (100 consumers) | (92 consumers) |
| Peformance | Expressed as % of the panel | |
| Reduced unsightly | 76 | 86 |
| excesses | | |
| Reduced unsightly | 70 | 80 |
| reserves | | |
| Refined silhouette | 75 | 87 |
| “Accelerates” the | 74 | 86 |
| removal of excesses | | |
| Models the body | 70 | 90 |
| Body contours better | 77 | 68 |
| defined | | |
| The silhouette is | 60 | — |
| resculptured | | |

1-19. (canceled)

20. A treatment method for promoting slimming, for fighting localized excess weight, refining the silhouette, accelerating the removal of excesses, better defining the contour of the body and/or resculpturing the silhouette, said method comprising applying a composition comprising one or more isoflavones in a glycone or glycosylated form.

21. The method of claim 20, wherein the composition is a topical composition.

22. The method of claim 20, wherein the composition is a cosmetic composition.

23. The method of claim 20, wherein applying a composition is directed to a skin of a patient.

24. The method of claim 20, wherein applying a composition is implemented topically.

25. The method of claim 20, wherein the isoflavone is genistein, daidzein, glycitein or a mixture thereof.

26. The method of claim 25, wherein the genistein is from soy.

27. The method of claim 20, further comprising applying one or two slimming agents of lipolytic type, one or two slimming agents of liporeducing type or a combination thereof.

28. The method of claim 27, wherein the agent of lipolytic type is caffeine, rhodysterol, palmitoylcarnitine, alpha bioactive agent, gamma bioactive agent, escin, ginkgo biloba or sphingosine.

29. The method of claim 27, wherein the agent of liporeducing type is andiroba, Garcinia cambogia or rutin.

30. The method of claim 20, further comprising applying one or two anti-filtration or venotonic agents, wherein said antifiltration or venotonic agent is viburnum, ivy, arnica, mouseear hawkweed, wild pansy, Fucus vesiculosus, Ruscus, Gingko biloba or escin.

31. The method of claim 20, further comprising applying an extract of Sophora japonica flowers, an extract of Centella asiatica, soy protein or any combination thereof.

32. A cosmetic composition, comprising one or more isoflavones in a glycone or glycosylated form and an extract of Sophora japonica flowers.

33. The cosmetic composition of claim 32, wherein the isoflavone is genistein, daidzein, glycitein or a mixture thereof.
